(19) United States
(12) Patent Application Publication
(10) Pub. No.: US 2005/0124721 A1
(43) Pub. Date: Jun. 9, 2005

(54) Bulky monomers leading to resins exhibiting low polymerization shrinkage

(76) Inventors: Samuel David Arthur, Wilmington, DE (US); Charles J. Brandenburg, Newark, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/936,106

(22) Filed: Sep. 8, 2004

Related U.S. Application Data
(60) Provisional application No. 60/527,103, filed on Dec. 3, 2003.

(51) Int. Cl. A61F 2/00; C07C 69/74

(52) U.S. Cl. 523/115; 560/1

(57) ABSTRACT

The invention relates to a dental composite material wherein space-filling compounds are utilized to reduce shrinkage upon polymerization; the invention also relates to a method for producing dental restoration articles with reduced shrinkage; the invention also relates to various dental restorative articles comprising the aforementioned space-filling compounds.

### FIELD OF THE INVENTION

This invention relates to composite materials for restorative dentistry. More particularly, it relates to a dental composite material that combines reduced shrinkage with sufficiently low viscosity, high polymerization rate, and good mechanical properties.

### BACKGROUND OF THE INVENTION

In recent years, composite materials comprising highly filled polymer have become commonly used for dental restorations. A thorough summary of current dental composite materials is provided in N. Moszner and U. Salz, Prog. Polym. Sci. 26:535-576 (2001). Currently used dental filling composites contain crosslinking acrylates or methacrylates, inorganic fillers such as glass or quartz, and a photoinitiator system, enabling them to be cured by radiation with visible light. Typical methacrylate materials include 2,2′-bis[4-(2-hydroxy-3-methacryloyloxypropyl)phenyl]propane (“Bis-GMA”); ethoxylated Bis-GMA (“EBPDMA”); 1,6-bis-[2-methacryloyloxyethoxycarbonylamino]-2,4,4-trimethylhexane (“UDMA”); dodecanediol dimethacrylate (“D₃MA”); and triethyleneglycol dimethacrylate (“TEGDMA”).

Dental composite materials offer a distinct cosmetic advantage over traditional metal amalgam. However, they do not offer the longevity of amalgam in dental fillings. The primary reasons for failure are believed to be excessive shrinkage during photopolymerization in the tooth cavity, which causes leakage and bacterial reentry, and inadequate strength and toughness.

The incumbent low-shrink monomer, Bis-GMA, the condensation product of bisphenyl A and glycidyl methacrylate, is an especially important monomer in dental composites. However, it is highly viscous at room temperature and consequently insufficiently converted to polymer. It is therefore typically diluted with a less viscous acrylate or methacrylate monomer, such as trimethylol propyl trimethacrylate, 1,6-hexanediol dimethacrylate, 1,3-butanediol dimethacrylate, ethylene glycol dimethacrylate, diethylene glycol dimethacrylate, TEGDMA, or tetraethylene glycol dimethacrylate. However, while providing fluidity, low molecular weight monomers contribute to increased shrinkage. Increasingly, Bis-GMA and TEGDMA have been combined with UDMA and ethoxylated-methacrylated versions of bisphenyl A, but shrinkage remains too high.

Increasing the amount of inorganic filler in the composite has moderated shrinkage. However, the amount of filler that can be added is severely limited by the resulting increase in viscosity. Also, it has been reported that the increase in modulus more than offsets this benefit and can lead to an increased build-up of stress during shrinkage. This “contraction stress” is of great importance in that it can lead to mechanical failure and debonding of the composite from the tooth, creating a gap that can permit microleakage of oral fluid and bacteria, causing a reinfection.

Another approach has been to prepolymerize the monomer, reducing the ultimate degree of polymerization and attendant shrinkage. However, this reduces the amount of inorganic filler that can be added below current levels, thus decreasing the modulus and other mechanical properties.

Spiro-type, “expanding” monomers, introduced in the 1970s, eliminate shrinkage, but they have never been commercialized because they polymerize too slowly and they, or their polymerization products, are too unstable. Diepoxide monomers are similarly limited in that they polymerize slowly for practical application, and the monomers and photosensitizers may be too toxic. They do not entirely eliminate shrinkage.

Slow cure and the so-called “soft start” photocure are also reported to reduce contraction stress.

Other systems have been reported in the literature but are not commercial. Liquid crystalline di(meth)acrylates shrink far less, but there is a tradeoff in mechanical properties. Branched polymethacrylates and so-called “macromonomers” offer lower viscosity at reduced shrinkage, but cost of manufacture may be excessive.

Published, unexamined Japanese Application JP2001122721 discloses tetramethylspirobisindanediol compounds wherein the benzene ring side chains comprise linear or branched (poly)oxyalkylenes with terminal (meth)acrylates.

U.S. Pat. No. 5,486,548 issued to Podszun et al. on Jan. 23, 1996, discloses di(meth)acrylate derivatives of cyclohexyldiphenyls that, when used in dental compositions, display a low degree of shrinkage upon polymerization.

B. Culbertson et al., Poly. Adv. Tech. 10:275-281 (1999) describes the synthesis and use of ethoxymethacrylate and propoxymethacrylate derivatives of fluorenylbisphenyl A.

U.S. Pat. No. 6,608,167 issued to Hayes et al. on Aug. 19, 2003, discloses a process for producing bis(2-hydroxyethyl)isosorbide.

There remains a need for a dental composite material that combines reduced shrinkage with sufficiently low viscosity, high polymerization rate, and acceptable mechanical properties.

### SUMMARY OF THE INVENTION

The present invention provides a dental composite material comprising at least one (meth)acrylic ester compound, at least one polymerization initiator, at least one inorganic filler, and at least one space-filling compound. The invention also provides a method of producing a dental restoration article using at least one (meth)acrylic ester compound, at least one polymerization initiator, at least one inorganic filler, and at least one space-filling compound.

Further disclosed is a method of treating dental tissue with a direct composite, comprising the steps of:
(a) placing a dental composite material, as desribed above, on a dental tissue;
(b) curing the dental composite material; and
(c) shaping the dental composite material.

### DETAILED DESCRIPTION OF THE INVENTION

Applicants specifically incorporate the entire content of all cited references in this disclosure. Applicants also incorporate by reference the co-owned and concurrently filed applications entitled “Dental Composites Containing Core-Shell Polymers with Low Modulus Cores” (Attorney Docket # CL 2434), “Dental Compositions Containing Liquid and Other Elastomers” (Attorney Docket # CL 2368), and “Branched Highly-Functional Monomers Exhibiting Low Polymerization Shrinkage” (Attorney Docket # CL 2452).

In the context of this disclosure, a number of terms shall be utilized.

The terms “(meth)acrylic” and “(meth)acrylate” as used herein denote “methacrylic or acrylic” and “methacrylate or acrylate” respectively.

The term “dental composite material” as used herein denotes a composition that can be used to remedy natural or induced imperfections of, and relating to, teeth. Examples include filling materials, reconstructive materials, restorative materials, crown and bridge materials, inlays, onlays, laminate veneers, dental adhesives, teeth, facings, pit and fissure sealants, cements, denture base and denture reline materials, orthodontic splint materials, and adhesives for orthodontic appliances.

Where a range of numerical values is recited herein, unless otherwise stated, the range is intended to include the endpoints thereof, and all integers and fractions within the range. It is not intended that the scope of the invention be limited to the specific values recited when defining a range.

The (meth)acrylic ester compound used in the present invention can comprise either a monofunctional compound or a polyfunctional compound which means a compound having one (meth)acrylic group and a compound having more than one (meth)acrylic group respectively. Specific examples of monofunctional (meth)acrylic ester compounds include methyl (meth)acrylate, ethyl (meth)acrylate, propyl (meth)acrylate, butyl (meth)acrylate, hydroxyethyl (meth)acrylate, benzyl (meth)acrylate, methoxyethyl (meth)acrylate, glycidyl (meth)acrylate, tetrahydrofurfuryl (meth)acrylate, and methacryloyloxyethyltrimellitic mono ester and its anhydride.

Specific examples of polyfunctional (meth)acrylic ester compounds include di(meth)acrylates of ethylene glycol derivatives as represented by the general formula 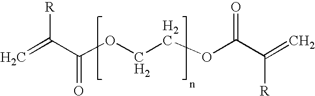
wherein R is hydrogen or methyl and n is an integer in a range of from 1 to 20, such as ethylene glycol di(meth)acrylate, diethylene glycol di(meth)acrylate, triethylene glycol di(meth)acrylate, and polyethylene glycol di(meth)acrylate; 1,3-butanediol di(meth)acrylate, 1,4-butanediol di(meth)acrylate, 1,6-hexanediol di(meth)acrylate, dodecanediol di(meth)acrylate, glycerol di(meth)acrylate, bisphenyl A di(meth)acrylate, bisphenyl A diglycidyl di(meth)acrylate and ethoxylated bisphenyl A diglycidyl di(meth)acrylate; urethane di(meth)acrylates; trimethylolpropane tri(meth)acrylate; tetrafunctional urethane tetra(meth)acrylates; pentaerythritol tetra(meth)acrylate, dipentaerythritol penta(meth)acrylate, and hexa(meth)acrylates of urethanes having an isocyanuric acid skeleton.

These (meth)acrylic ester compounds may be used alone or in admixture of two or more. The mixtures can be mixtures of monofunctionals, polyfunctionals, or both.

The (meth)acrylic ester compound used in the dental compositions preferably comprises at least one polyfunctional (meth)acrylic ester compound, and more preferably comprises at least two polyfunctional (meth)acrylic ester compounds.

The space-filling compound of the present invention is a monomer comprising a rigid, angular, bulky moiety that can be compounded into composites, which upon polymerization exhibit low volumetric shrinkage. By “space-filling compound” is meant a monomer comprising a moiety with an inability of a significant fraction of its constituent atoms to be place in a common plane. By “significant fraction” is meant greater than about 15%. Additionally, the constituent atoms have a relative lack of mobility with respect to one another; that is, the moiety's structure is highly rigid and preferably has less than two freely rotating internal bonds.

In accordance with one aspect of the invention, the space-filling compounds comprise derivatives of at least one of the moieties spirobisindanediol (“SBID”), phenylindane dicarboxylic acid (“PIDA”), t-butylisophthalic acid (“BIPA”), cyclohexyldiphenyl, fluorenylbisphenyl A, tetrahydrodicyclopentadiol, phenyl-alkyl levulinate, and isosorbide.

Preferred SBID-based space-filling compounds comprise at least one of compound (i) or (ii): 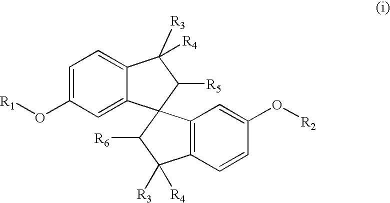
wherein R₁ and R₂ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacryl ate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; R₃ and R₄ are independently H, CH₃, alkyl, or aralkyl such that the carbon atom attached to the cyclopentane ring is aliphatic with at least one H (i.e., —CHR—); and R₅ and R₆ are independently H, CH₃, alkyl, or aralkyl, containing one carbon less than R₃ and R₄ respectively; provided when R₃ and R₄ are CH₃ and R₅ and R₆ are H that R₁ and R₂ are independently 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate. Preferably, R₁ and R₂ are 2-(2-ethoxycarbonylamino)ethyl methacrylate, R₃ and R₄ are CH₃, and R₅ and R₆ are H; and 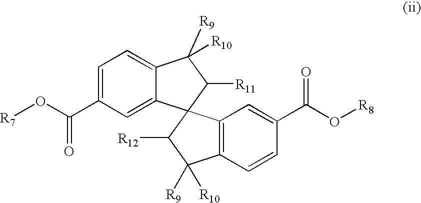
wherein R₇ and R₈ are independently 2-acryloyloxyethyl; 2-methacryloyloxyethyl; 2-acryloyloxypropyl; 2-methacryloyloxypropyl; 3-acryloyloxy-2,2-dimethylpropyl; 3-methacryloyloxy-2,2-dimethylpropyl; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[3-(2,2-dimethylpropoxy)carbonylamino]ethyl acrylate; 2-[3-(2,2-dimethylpropoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; R₉ and R₁₀ are independently H, CH₃, alkyl, or aralkyl such that the carbon atom attached to the cyclopentane ring is aliphatic with at least one H (i.e., —CHR—); and R₁₁ and R₁₂ are independently H, CH₃, alkyl, or aralkyl, containing one carbon less than R₉ and R₁₀ respectively. Preferably, R₇ and R₈ are 2-[3-(2,2-dimethylpropoxy)carbonylamino]ethyl methacrylate, R₉ and R₁₀ are CH₃, and R₁₁ and R₁₂ are H.

Preferred PIDA-based space-filling compounds (iii) comprise 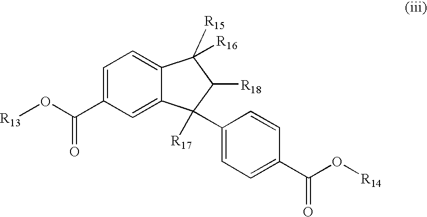
wherein R₁₃ and R₁₄ are independently 2-acryloyloxyethyl; 2-methacryloyloxyethyl; 2-acryloyloxypropyl; 2-methacryloyloxypropyl; 3-acryloyloxy-2,2-dimethylpropyl; 3-methacryloyloxy-2,2-dimethylpropyl; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[3-(2,2-dimethylpropoxy)carbonylamino]ethyl acrylate; 2-[3-(2,2-dimethylpropoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; R₁₅, R₁₆, and R₁₇ are independently H, CH₃, alkyl, or aralkyl such that the carbon atom attached to the cyclopentane ring is aliphatic with at least one H (i.e., —CHR—); and R₁₈ is H, CH₃, alkyl, or aralkyl, containing one carbon less than R₁₅, R₁₆, or R₁₇. Preferably, R₁₃ and R₁₄ are 2-[3-(2,2-dimethylpropoxy)carbonylamino]ethyl methacrylate, R₁₅, R₁₆, and R₁₇ are CH₃ and R₁₈ is H.

Preferred phenyl-alkyl levulinate-based space-filling compounds (iv) comprise 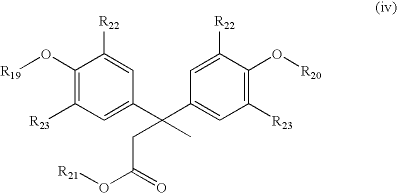
wherein R₁₉ and R₂₀ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; R₂₁, R₂₂, and R₂₃ are independently H, CH₃, alkyl, or aralkyl. Preferably, R₁₉ and R₂₀ are 2-(2-ethoxycarbonylamino)ethyl methacrylate, R₂₁ is ethyl and R₂₂ and R₂₃ are H.

Preferred cyclohexyldiphenyl-based space-filling compounds (v) comprise 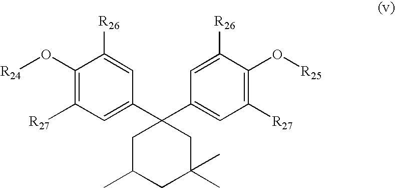
wherein R₂₄ and R₂₅ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; and R₂₆ and R₂₇ are independently H, CH₃, alkyl, or aralkyl; provided when R₂₆ and R₂₇ are H that R₂₄ and R₂₅ are independently 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate. Preferably, R₂₄ and R₂₅ are 2-(2-ethoxycarbonylamino)ethyl methacrylate and R₂₆ and R₂₇ are H.

Preferred fluorenylbisphenyl A-based space-filling compounds (vi) comprise 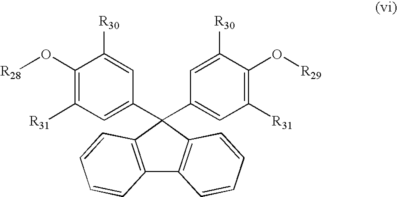
wherein R₂₈ and R₂₉ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; and R₃₀ and R₃₁ are independently H, CH₃, alkyl, or aralkyl; provided when R₃₀ and R₃₁ are H or CH₃ that R₂₈ and R₂₉ are independently 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate. Preferably, R₂₈ and R₂₉ are 2-(2-ethoxycarbonylamino)ethyl methacrylate and R₃₀ and R₃₁ are H.

Preferred tetrahydrodicyclopentadiol-based space-filling compounds (vii) comprise 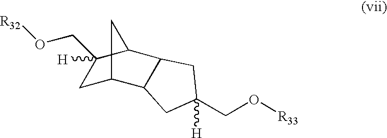
wherein R₃₂ and R₃₃ are independently 2-(carbonylamino)ethyl acrylate; 2-(carbonylamino)ethyl methacryl ate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate. Preferably, R₃₂ and R₃₃ are 2-(2-ethoxycarbonylamino)ethyl methacrylate.

Preferred isosorbide-based space-filling compounds (viii) comprise 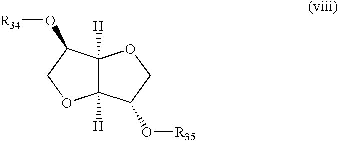
wherein R₃₄ and R₃₅ are independently 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate. Preferably, R₃₄ and R₃₅ are 2-(2-ethoxycarbonylamino)ethyl methacrylate.

Preferred BIPA-based space-filling compounds (ix) comprise 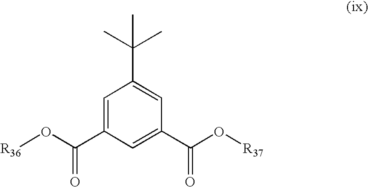
wherein R₃₆ and R₃₇ are independently 2-acryloyloxyethyl; 2-methacryloyloxyethyl; 2-acryloyloxypropyl; 2-methacryloyloxypropyl; 3-acryloyloxy-2,2-dimethylpropyl; 3-methacryloyloxy-2,2-dimethylpropyl; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[3-(2,2-dimethylpropoxy)carbonylamino]ethyl acrylate; 2-[3-(2,2-dimethylpropoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate. Preferably, R₃₆ and R₃₇ are 2-[3-(2,2-dimethylpropoxy)carbonylamino]ethyl methacrylate.

Monomers of diol-based space-filling compounds can be reacted with ethylene or propylene oxide, for example, to produce low molecular weight alkoxylate oligomers that can then be (meth)acrylated to produce free radical-polymerizable monomers. Monomers of dicarboxylic acid-based space-filling compounds can be esterfied with diols, for example, to produce low molecular weight esterdiol oligomers that can then be (meth)acrylated to produce free radical-polymerizable monomers.

In another aspect of the invention, dental composite materials comprise a space-filling compound that has been functionally terminated with at least two urethane (meth)acrylate groups. Preferably, the space-filling compound is functionally terminated with 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; 2-(omega-polyoxypropylenecarbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[3-(2,2-dimethylpropoxy)carbonylamino]ethyl acrylate; or 2-[3-(2,2-dimethylpropoxy)carbonylamino]ethyl methacrylate.

In dental composite materials, space-filling compounds of the present invention can be used in the range of about 1 weight percent to 100 weight percent, preferably in the range of about 20 weight percent to about 80 weight percent, and more preferably in the range of about 40 weight percent to about 60 weight percent, the percentages being based on the total weight exclusive of filler.

The production of the crosslinked polymers useful in the practice of this invention from monomers and crosslinking agents may be performed by any of the many processes known to those skilled in the art. Thus, the polymers may be formed by heating a mixture of the components to a temperature sufficient to cause polymerization. For this purpose, peroxy-type initiators such as benzoyl peroxide, dicumyl peroxide, lauryl peroxide, tributyl hydroperoxide, and other materials familiar to those skilled in the art may be employed, and the use of activators may be advantageous in some formulations. Suitable activators include, for example, N,N-bis-(hydroxyalkyl)-3,5-xylidines, N,N-bis-(hydroxyalkyl)-3,5-di-t-butylanilines, barbituric acids and their derivatives, and malonyl sulfamides, including specific examples of these activators found in published U.S. Patent Application 2003/0008967. Azo-type initiators such as 2,2′-azobis(isobutyronitrile), 2,2′-azobis(2,4-dimethyl valeronitrile), 2,2′-azobis(2-methyl butane nitrile), and 4,4′-azobis(4-cyanovaleric acid) may also be used. Alternatively, the crosslinked polymers of the invention may be formed from the constituents by photochemical or radiant initiation utilizing light or high energy radiation. For photochemical initiation, photochemical sensitizers, or energy transfer compounds may be employed to enhance the overall polymerization efficiency in manners well known to those skilled in the art.

Suitable photoinitiators include, for example, camphor quinone, benzoin ethers, α-hydroxyalkylphenones, acylphosphine oxides, α,α-dialoxyacetophenones, α-aminoalkylphenones, acyl phosphine sulfides, bis acyl phosphine oxides, phenylglyoxylates, benzophenones, thioxanthones, metallocenes, bisimidazoles, and α-diketones.

Photoinitiating accelerators may also be present. Such photoinitiating accelerators include, for example, ethyl dimethylaminobenzoate, dimethylaminoethyl methacrylate, dimethyl-p-toluidine, and dihydroxyethyl-p-toluidine.

According to another aspect, an inorganic filler is included in the composite. Included in the inorganic fillers are the preferred silicious fillers. More preferred are the inorganic glasses. Among these preferred inorganic fillers are barium aluminum silicate, lithium aluminum silicate, strontium fluoride, lanthanum oxide, zirconium oxide, bismuth phosphate, calcium tungstate, barium tungstate, bismuth oxide, tantalum aluminosilicate glasses, and related materials. Glass beads, silica, especially in submicron sizes, quartz, borosilicates, alumina, alumina silicates, and other fillers may also be employed. For example, Aerosil® OX-50 fumed silica from Degussa can be used. Mixtures of fillers may also be employed. The average diameter of the inorganic fillers is preferably less than 15 μm, even more preferably less than 10 μm.

Such fillers may be silanated prior to use in this invention. Silanation is well known to those skilled in the art and any silanating compound known to them may be used for this purpose. By “silanation” is meant that some of the silanol groups have been substituted or reacted with, for example, dimethyldichlorosilane to form a hydrophobic filler. The particles are typically from 50 to 95 percent silanated. Silanating agents for inorganic fillers include, for example, γ-mercaptoproyltrimethoxysilane, γ-mercaptopropyltriethoxysilane, γ-aminopropyltriethoxysilane, γ-methacryloyloxypropyltrimethoxysilane, and γ-methacryloyloxypropyltriethoxysilane.

The (meth)acrylic ester compound can be used in the range of about 1 weight percent to about 99 weight percent, preferably in the range of about 20 weight percent to about 80 weight percent, and more preferably in the range of about 40 weight percent to about 60 weight percent, the percentages being based on the total weight exclusive of filler.

The polymerization initiator with, optionally, a photoinitiating accelerator can be used in the range of about 0.1 weight percent to about 5 weight percent, preferably in the range of about 0.2 weight percent to about 3 weight percent, and more preferably in the range of about 0.2 weight percent to about 2 weight percent, the percentages being based on the total weight exclusive of filler.

The inorganic filler can be used in the range of about 20 weight percent to about 90 weight percent, preferably in the range of about 40 weight percent to about 90 weight percent, and more preferably in the range of about 50 weight percent to about 85 weight percent, the percentages being based on the total weight of the (meth)acrylic ester compound, the polymerization initiator, the inorganic filler, and the space-filling compound.

In addition to the components described above, the blend may contain additional, optional ingredients. These may comprise activators, pigments, radiopaquing agents, stabilizers, antioxidants, and other materials as will occur to those skilled in the art.

Suitable pigments include, for example, inorganic oxides such as titanium dioxide, micronized titanium dioxide, and iron oxides; carbon black; azo pigments; phthalocyanine pigments; quinacridone pigments; and pyrrolopyrrol pigments.

Preferred radiopaquing agents include, for example, ytterbium trifluoride, yttrium trifluoride, barium sulfate, bismuth subcarbonate, bismuth trioxide, bismuth oxichloride, and tungsten.

Preferred stabilizers can include, for example, hydroquinone, hydroquinone monomethyl ether, 4-tert-butylcatechol, and 2,6-di-tert-butyl-4-methylphenyl.

Primary antioxidants, secondary antioxidants, and thioester-type antioxidants are all suitable for use in the dental compositions of the invention. Preferred primary antioxidants comprise hindered phenyl and amine derivatives such as butylated hydroxytoluene, butylated hydroxyanisole, t-butyl hydroquinone, and α-tocopherol. Preferred secondary antioxidants include phosphites and phosphonites such as tris(nonylphenyl) phosphite, tris(2,4-di-t-butylphenyl) phosphite, distearyl pentaerythritol diphosphite, bis(2,4-dicumylphenyl) pentaerythritol diphosphite, and Irgafos® P-EPQ (Ciba Specialty Chemicals, Tarrytown, N.Y.). Preferred thioester-type antioxidants, used synergistically or additively with primary antioxidants, include dilauryl 3,3′-thiodipropionate, dimyristyl 3,3′-thiodipropionate, distearyl 3,3′-thiodipropionate, and ditridecyl 3,3′-thiodipropionate.

Organic fillers, comprising prepolymerized material, optionally comprising at least one of the (meth)acrylic ester compounds and space-filling compounds, and optionally comprising inorganic filler, may also be included in the composite material. Prepolymerization filler can be produced by any method known in the art, for example, by the method described in published U.S. patent application 2003/0032693. Optionally, uniformly-sized bead methacrylate polymers, such as Plexidon® or Plex® available from Röhm America LLC (Piscataway, N.J.), may be utilized as organic fillers.

The dental composite materials of the present invention can be used in any treatment method known to one of ordinary skill in the art. Treatment in this context includes preventative, restorative, or cosmetic procedures using the dental composites of the present invention. Typically, without limiting the method to a specific order of steps, the dental composite materials are placed on a dental tissue, either natural or synthetic, the dental composite materials are cured by any method known to one of ordinary skill in the art, and the dental composite materials are shaped as necessary to conform with the target dental tissue. Dental tissue includes, but is not limited to, enamel, dentin, cementum, pulp, bone, and gingiva.

The dental composite materials of the present invention are suitable for a very wide range of dental uses, including fillings, teeth, bridges, crowns, inlays, onlays, laminate veneers, facings, pit and fissure sealants, cements, denture base and denture reline materials, orthodontic splint materials, and adhesives for orthodontic appliances. The materials of the invention may also be utilized for prosthetic replacement or repair of various hard body structures such as bone and may be utilized for reconstructive purposes during surgery, especially oral surgery. They are also useful for various non-dental uses as, for example, in plastic construction materials.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations is as follows: “hr.” means hour(s), “min.” means minute(s), “sec.” means second(s), “ml” means milliliter(s), “cm” means centimeter(s), “mm” means millimeter(s), “g” means gram(s), “mmol” means millimole(s), “wt %” means weight percent(age), “mW” means milliwatt(s), “atm.” means atmosphere(s), “Mₙ” means number average molecular weight, “MPa” means megapascal(s), “d50” means 50% of particles have a diameter below a given size, “MEHQ” means 4-methoxyphenyl, “PTFE” means polytetrafluoroethylene, “TH F” means tetrahydrofuran.

### Example 1

### Bis-GMA/TEGDMA Glass Composition

A masterbatch containing 15.0 g Bis-GMA (Sigma-Aldrich, St. Louis, Mo.), 15.0 g TEGDMA (Sigma-Aldrich), 0.40 g camphor quinone (Sigma-Aldrich), and 0.40 g ethyl 4-N,N-dimethylaminobenzoate (Sigma-Aldrich) was made up by mixing the components well under subdued light. Then, 5.0 g of this masterbatch was combined and mixed well with 1.0 g untreated Degussa OX-50 fumed silica followed by 14.0 g Schott 8235 UF1.5 (d50=1.5 micron) glass powder coated with 2.3 wt % trimethoxysilylpropyl methacrylate. The blend was then placed on a PTFE sheet and mixed by folding over and flattening out the doughy composition 60 times. The resin-glass mixture was degassed under 40 mm Hg vacuum for 18 hr. at room temperature followed by heating in a vacuum oven at 45° C. with very slight vacuum for an additional 16 hr. This composition contained 25.0 wt % resin, 5.0 wt % fumed silica, and 70.0 wt % glass.

### Example 2

### Synthesis of Tetramethylspirobisindanediol (“SBID”)

A mixture of 500 g bisphenyl A and 1000 ml 48% aqueous hydrobromic acid was stirred at reflux under nitrogen overnight (about 16 hrs.) in a 2 l 3-neck flask with overhead stirrer and reflux condenser. The mixture was cooled to room temperature, and the upper red phase, which contained the product, solidified. The hydrobromic acid was decanted off, and the solid product was crushed and washed on a fritted filter funnel with water until the washes were neutral to pH paper.

The product was taken up in 500 ml boiling methanol and precipitated by addition of 700 ml water to the boiling solution. Suction filtration of the thick slurry yielded a tan powder that was taken up in 600 ml boiling methanol. Addition of 100 ml water just started to cause precipitation, so 10-20 ml methanol was added to form a clear solution, and then the mixture was cooled in ice. The solids were suction filtered and taken up in 400 ml boiling methanol. This solution was cooled in ice, the resulting slurry was suction filtered, and the solids were washed with 2×100 ml methanol. Air-drying on the funnel yielded 67 g tetramethylspirobisindanediol (“SBID”). The filtrate was evaporated down to about half its volume and chilled in ice. Suction filtration yielded 72 g less pure SBID.

The resulting compound has the formula:

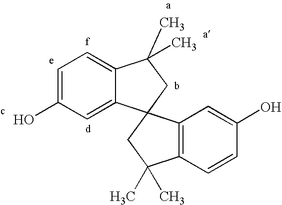

Lower case letters refer to ¹H NMR (CDCl₃; sparingly soluble) results as follows: 1.31 ppm (s, a, 3H); 1.36 (s, a′, 3H); 2.22 (d, J=13.1 Hz, b, 1H); 2.32 (d, J=13.1 Hz, b, 1H); 4.38 (s, C, 1H); 6.20 (d, J=2.3 Hz, d, 1H); 6.68/6.71 (d of d, J=2.4, 8.2 Hz, e, 1H); 7.02 (d, J=8.2 Hz, f, 1H).

### Example 3

### Synthesis of Tetramethylspirobisindane Bis(2-Hydroxyethyl Ether) (“SBID EO”)

A 5.0 g sample (16 mmol; 32 mmol OH) of SBID from Example 2 was dissolved in 50 ml MeOH. The hazy solution was clarified through a 5-micron syringe filter and combined with 0.5 g (4.5 mmol) potassium t-butoxide in a 100 ml RB flask. A dry ice condenser and gas inlet were attached to the flask containing the pink solution, and 6.5 g (150 mmol) ethylene oxide (“EO”) was condensed into the flask. The solution became warm upon introduction of the EO. The solution was stirred at reflux under nitrogen in a 70° C. water bath for 4 hr. The solution was allowed to stand at room temperature overnight and was then rotovapped to give a white-pink solid. The powdery solid was suspended in 50 ml water, acidified with aqueous HCl, and stirred for 30 min. The suspension was suction filtered, water washed to neutral pH, and air dried under suction to yield 6.05 g off-white powder tetramethylspirobisindane bis(2-hydroxyethyl ether) (“SBID EO”).

The resulting compound has the formula:

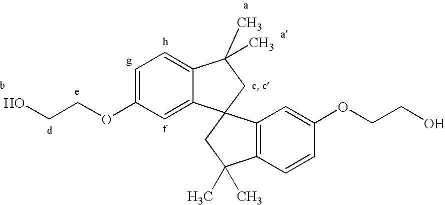

Lower case letters refer to ¹H NMR (CDCl₃) results as follows: 1.32 ppm (s, a, 3H); 1.38 (s, a′, 3H); 2.02 (t, J=6.4 Hz, b, 1H); 2.24 (d, J=13.1 Hz, c, 1H); 2.34 (d, J=13.1 Hz, c′, 1H); 3.86 (q, J=4.9 Hz, d, 2H); 3.97 (t, J=4.6 Hz, e, 2H); 6.34 (d, J=2.4 Hz, f, 1H); 6.79/6.80 (d of d, J=2.6, 8.2 Hz, g, 1H); 7.07 (d, J=8.2 Hz, h, 1H).

There were also several small triplets due to impurities at 2.11, 2.17, 3.61, 3.78, and 4.02 ppm as well as a quartet at 3.69. The impurities are due to multiple EO additions.

### Example 4

### Synthesis of Tetramethylspirobisindane Bis[2-(2-Ethoxycarbonylamino)ethyl Methacrylate] (“SBID EOUMA”)

A mixture of 3.0 g (15 mmol OH) SBID EO from Example 3, 1 drop of dibutyltin diacetate, 10 mg MEHQ, and 2.7 g (17 mmol) 2-isocyanatoethyl methacrylate in 20 ml THF in a 200 ml RB flask was stirred in a 60° C. oil bath for 1 hr.

The light tan solution was quickly rotovapped to remove over half of the solvent, and the liquid concentrate was stirred with 100 ml hexane for 1 hr. The hexane was decanted from the taffy-like product, and 100 ml fresh hexane was added. The mixture was stirred for 1 hr., and the hexane was decanted off. A solution of 10 mg MEHQ in 2 ml dichloromethane was added and mixed well. The solution was held under vacuum with an air bleed to remove solvent, yielding 5.68 g tetramethylspirobisindane bis[2-(2-ethoxycarbonylamino)ethyl methacrylate] (“SBID EOUMA”). NMR indicated complete conversion of the SBID EO hydroxyls to urethane methacrylate groups, the OH peak at 2.02 ppm having been replaced by the methacrylate methyl at 1.92 ppm.

The resulting compound has the formula:

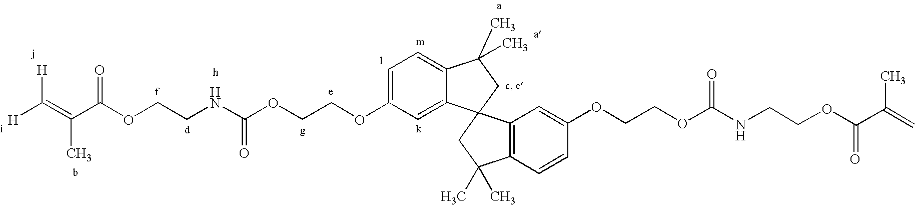

Lower case letters refer to ¹H NMR (CDCl₃) results as follows: 1.31 ppm (s, a, 3H); 1.37 (s, a′, 3H); 1.92 (s, b, 3H); 2.23 (d, J=13.1 Hz, c, 1H); 2.33 (d, J=13.1 Hz, c′, 1H); 3.48 (br m, d, 2H); 4.04 (t, e, 2H); 4.20 (t, f, 2H); 4.35 (t, g, 2H); 5.09 (t, h, <1H); 5.56 (s, i, 1H); 6.09 (s, j, 1H); 6.32 (d, J=2.2 Hz, k, 1H); 6.77/6.79 (d of d, J=2.6, 8.2 Hz, l, 1H); 7.07 (d, J=8.2 Hz, m, 1H).

### Example 5

### SBID EOUMA/TEGDMA—Glass Composition

A TEGDMA/photoinitiator masterbatch was produced by combining 10.0 g TEGDMA with a solution of 0.20 g phenylbis(2,4,6-trimethylbenzoyl)phosphine oxide (Sigma-Aldrich) in 0.5 ml dichloromethane. The flask was covered with foil, and the solution magnetically stirred under 10-20 mm Hg vacuum for 1 hr. with an air bleed to carry off solvent.

A mixture of 1.25 g TEGDMA/photoinitiator masterbatch and 1.25 g SBID EOUMA from Example 4 was combined in a scintillation vial and mixed with a spatula to a uniform mixture. Then, 0.50 g Degussa OX-50 fumed silica was mixed in with a spatula, followed by 7.0 g silanated Schott 8235 UF1.5 glass powder. The blend was placed on a PTFE sheet and mixed by folding over and flattening out the doughy composition 40 times. The glass-resin blend was held under 40 mm Hg vacuum at room temperature for 16 hrs. and then in an oven at 45° C. under 1 atm. air for 24 hrs. This composition contained 25.0 wt % resin, 5.0 wt % fumed silica, and 70.0 wt % glass. The resin-glass blend was molded and cured into bars for physical testing as described below in Example 6.

### Example 6

Fracture toughness (K_{IC}), flexural strength (ISO 4049), and density were determined on molded and cured bars of the resin composition (Bis-GMA/TEGDMA from Example 1 and SBID EOUMA/TEGDMA from Example 5). Bars (2 mm×2 mm×25 mm) were molded and cured by irradiating 2 min. on a side using an array of three Denstply Spectrum 800 dental lamps at 800 mW/cm². The metal mold was covered on both sides with a 3-mil polyester film to exclude oxygen, which would inhibit cure.

The fracture toughness test was based on both the ASTM polymers standard (ASTM D5045) and the ASTM ceramics standard (ASTM C1421, precracked beam method). Testing was conducted at a test speed of 0.5 mm/min. at room temperature and ambient humidity using a three-point bend fixture (span to depth ratio of 10). The specimens were molded using the flex bar mold specified in ISO 4049. The specimens were precracked halfway through the depth. Two modifications to the test procedures were made. The first was the use of smaller test specimens than those recommended in the ASTM C1421 standard (2 mm×2 mm×25 mm instead of the recommended minimum dimensions of 3 mm×4 mm×20 mm). The second was the use of a slitting circular knife to machine the precracks. The knife was 0.31 mm in thickness with a 9 degree single bevel. Tests have shown that this technique produced precracks that were equivalent to precracks produced using techniques recommended in ASTM D5045.

Density determination was accomplished via helium pycnometry. The densities of the uncured glass-resin blends were determined as well.

Polymerization shrinkage was determined by the equation: [(ρ_{cured}−ρ_{uncured})/(ρ_{cured})]×100%=% S.

As seen in Table 1, use of the bulky monomer with the spirobisindane structure reduced polymerization shrinkage by over 25% relative to the bisphenyl A monomer control composition without significantly reducing mechanical properties.

TABLE 1

| Resin Mixture | | SBID EOUMA/ |
|---|---|---|
| (1:1) | Bis-GMA/TEGDMA | TEGDMA |
| Shrinkage, % | 4.56 | 3.37 |
| K_{IC}, MPa · m^{1/2} | 1.88 | 1.69 |
| Flex Strength, | 118 | 129 |
| MPa · m^{1/2} | | |

### Example 7

### Synthesis of Tetramethylspirobisindane Bis(2-Hydroxypropyl Ether) (“SBID PO”)

A 5.0 g sample (32.5 mmol OH) of SBID from Example 2 was combined with 0.1 g 2-methylimidazole and 3.5 ml (4.2 g; 41 mmol) propylene carbonate in a 100 ml RB flask under nitrogen. The dark, fluid homogeneous melt was magnetically stirred in a 180° C. oil bath for 5 hrs., and then 75 ml water was added slowly down the condenser. This mixture was stirred at reflux for 15 mins., the flask was then cooled, and the water decanted off. The solid product was broken up, and 75 ml fresh water was added. The mixture was stirred at reflux for another 15 min. The suspension was cooled and suction filtered dry to yield 6.76 g tetramethylspirobisindane bis(2-hydroxypropyl ether) (“SBID PO”). NMR indicated clean conversion to PPO diadduct.

The resulting compound has the formula:

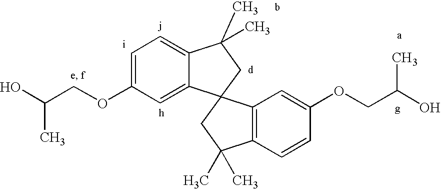

Lower case letters refer to ¹H NMR (CDCl₃) results as follows: 1.21/1.23 ppm (s, a, 6H); 1.32/1.38 (s, b, 12H); 1.63/2.31 (br s, J=268 Hz, c, 2H); 2.24 (d, J=13.1 Hz, d, 2H); 2.34 (d, J=13.1 Hz, d, 2H); 3.68 (m, e, 2H); 3.83 (d of d, f, 2H); 4.11 (m, g, >1.5H); 4.36 (m, g, <0.5H; may be opposite addition of propylene carbonate); 6.33 (d, J=2.4 Hz, h, 2H); 6.79/6.80 (d of d, J=2.6, 8.2 Hz, i, 1H); 7.08 (d, J=8.2 Hz, h, 2H).

### Example 8

### Synthesis of Bisphenyl A Bis(2-Hydroxypropyl Ether) (“BPA PO”)

A 6.0 g sample (52.6 mmol OH) of bisphenyl A was combined with 0.1 g 2-methylimidazole and 6.0 ml (7.1 g; 70 mmol) propylene carbonate in a 100 ml RB flask under nitrogen. The homogeneous melt was magnetically stirred in a 180° C. oil bath for 5 hrs., and then 75 ml water as added slowly down the condenser. This mixture was stirred at reflux or 15 mins., the flask cooled, and the water decanted off and replaced by 75 ml fresh water. The fluid product was stirred at reflux for another 15 min. and cooled, and the water was decanted off again. The product was held under high vacuum in a boiling water bath for 2 hrs. to yield 8.92 g bisphenyl A bis(2-hydroxypropyl ether) (“BPA PO”). NMR indicated clean conversion to PPO diadduct. There also appeared to be a little PPO oligomer (1.13 ppm) present.

The resulting compound has the following formula:

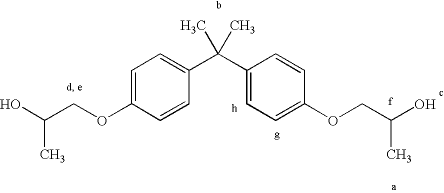

Lower case letters refer to ¹H NMR (CDCl₃) results as follows: 1.25/1.27 ppm (s, a, 6H); 1.63 (s, b, 6H); 2.41 (br s, c, ˜2H); 3.75/3.78 (d of d, J=7.6 Hz, d, 2H); 3.89/3.92 (d of d, J=3.3 Hz, e, 2H); 4.16 (m, f, >1.5H); 4.45 (m, f, <0.5H; may be opposite addition of propylene carbonate); 6.80 (d, J=8.8 Hz, g, 4H); 7.13 (d, J=8.8 Hz, h, 4H).

### Example 9

### Synthesis of Tetramethylspirobisindane Bis(2-Hydroxylpropyl Ether) Dimethacrylate (“SBID POMA”)

A mixture of 5.0 g (11.8 mmol; 23.6 mmol OH) SBID PO from Example 7, 10.0 g (65 mmol) methacrylic anhydride, and 2.0 g (25 mmol) pyridine was stirred in a 50 ml RB flask under air in a 120° C. oil bath for 5 hrs. The solution was cooled to room temperature, added to 100 ml water containing 8 g sodium carbonate, and stirred for 30 mins. The aqueous mixture was briefly shaken in a separatory funnel with 50 ml diethyl ether. The water was separated, and the ether was shaken briefly with 25 ml of water containing 5 ml concentrated HCl. The acidic water was again separated, and the ether layer was shaken briefly with 20 ml of water containing 2 g sodium carbonate. The ether was separated and dried over magnesium sulfate followed by filtration; 5 mg MEHQ was added to the filtrate. The solution was quickly rotovapped from warm water then held under 20 mm Hg vacuum overnight with an air bleed through a syringe needle to yield 7.04 g tetramethylspirobisindane bis(2-hydroxylpropyl ether) dimethacrylate (“SBID POMA”).

¹H NMR (CDCl₃) indicated 80% conversion to dimethacrylate. The ratio of the integrals of the 5.55 ppm methacrylate vinyl proton to the 7.10 ppm aromatic ring proton equaled 0.80.

### Example 10

### Synthesis of Bisphenyl A Bis(2-Hydroxypropyl Ether) Dimethacrylate (“BPA POMA”)

A mixture of 3.8 g (11 mmol; 22 mmol OH) BPA PO from Example 8, 5.0 g (32 mmol) methacrylic anhydride, and 2.0 g (24 mmol) pyridine was stirred in a 50 ml RB flask under air in a 120° C. oil bath for 5 hrs. IR of a sample showed the absence of OH at 3,400-3,500 cm⁻¹ as well as a strong 1,720 cm⁻¹ ester peak.

The mixture was added to 30 ml water containing 1 g sodium carbonate and stirred for 30 mins. followed by extraction with 50 ml diethyl ether. The ether layer was separated and washed with 10 ml water containing 1 ml concentrated HCl, separated again, washed with 5 ml 5% aqueous sodium bicarbonate, and dried over magnesium sulfate. The ether was filtered, and 5 mg MEHQ was added to the filtrate. The solution was quickly rotovapped from hot water and then held under 20 mm Hg vacuum overnight with an air bleed through a syringe needle to yield 4.25 g bisphenyl A bis(2-hydroxypropyl ether) dimethacrylate (“BPA POMA”).

NMR (CDCl₃) indicated 85-90% conversion to methacrylate diester by ratio of aromatic ring protons (7.10 ppm) to methacrylate vinyl protons (6.08 ppm).

### Example 11

### SBID POMA/TEGDMA—Glass Composition

A mixture of 1.25 g TEGDMA/photoinitiator masterbatch from Example 5 and 1.25 g SBID POMA from Example 9 was combined in a scintillation vial and mixed with a spatula to a uniform mixture. Then, 0.50 g Degussa OX-50 fumed silica was mixed in with a spatula, followed by 7.0 g silanated Schott 8235 UF1.5 glass powder. The blend was placed on a PTFE sheet and mixed by folding over and flattening out the doughy composition 40 times. The glass-resin blend was held under 40 mm Hg vacuum at room temperature for 16 hr. and then in an oven at 50° C. under 17″ vacuum (330 mm Hg) with an air bleed for 8 hr. This composition contained 25.0 wt % resin, 5.0 wt % fumed silica, and 70.0 wt % glass. The resin-glass blend was molded and cured into bars for physical testing as described in Example 6.

### Example 12

### BPA POMA/TEGDMA—Glass Compostion

A mixture of 1.25 g TEGDMA/photoinitiator masterbatch from Example 5 and 1.25 g BPA POMA from Example 10 was combined in a scintillation vial and mixed with a spatula to a uniform mixture. Then, 0.50 g Degussa OX-50 fumed silica was mixed in with a spatula, followed by 7.0 g silanated Schott 8235 UF1.5 glass powder. The blend was placed on a PTFE sheet and mixed by folding over and flattening out the doughy composition 40 times. The glass-resin blend was held under 40 mm Hg vacuum at room temperature for 16 hr. and then in an oven at 50° C. under 17″ vacuum (330 mm Hg) with an air bleed for 8 hr. This composition contained 25.0 wt % resins, 5.0 wt % fumed silica, and 70.0 wt % glass. The resin-glass blend was molded and cured into bars for physical testing as described in Example 6.

### Example 13

Physical tests were performed on the SBID POMA/TEGDMA bars from Example 11 and the BPA POMA/TEGDMA bars from Example 12 as described in Example 6.

As seen in Table 2, use of the bulky monomer with the spirobisindane structure reduced polymerization shrinkage by 15% relative to the bisphenyl A monomer control composition without significantly compromising mechanical properties.

TABLE 2

| Resin Mixture | BPA POMA/ | SBID POMA/ |
|---|---|---|
| (1:1) | TEGDMA | TEGDMA |
| Shrinkage, % | 4.86 | 4.13 |
| K_{IC}, MPa · m^{1/2} | 1.67 | 1.56 |
| Flex Strength, | 138 | 102 |
| MPa · m^{1/2} | | |

1. A compound of the formula 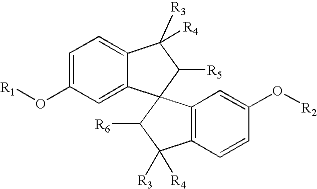
wherein
R₁ and R₂ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₃ and R₄ are independently H, CH₃, alkyl, or aralkyl such that the carbon atom attached to the cyclopentane ring is aliphatic with at least one H; and
R₅ and R₆ are independently H, CH₃, alkyl or aralkyl, containing one carbon less than R₃ and R₄ respectively;
provided when R₃ and R₄ are CH₃ and R₅ and R₆ are H that R₁ and R₂ are independently 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate.

2. A compound of the formula 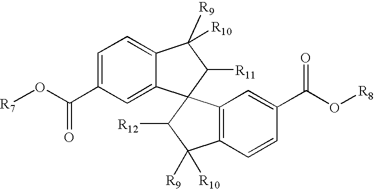
wherein
R₇ and R₈ are independently 2-acryloyloxyethyl; 2-methacryloyloxyethyl; 2-acryloyloxypropyl; 2-methacryloyloxypropyl; 3-acryloyloxy-2,2-dimethylpropyl; 3-methacryloyloxy-2,2-dimethylpropyl; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[3-(2,2-dimethylpropoxy)carbonylamino]ethyl acrylate; 2-[3-(2,2-dimethylpropoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₉ and R₁₀ are independently H, CH₃, alkyl, or aralkyl such that the carbon atom attached to the cyclopentane ring is aliphatic with at least one H;
R₁₁ and R₁₂ are independently H, CH₃, alkyl, or aralkyl, containing one carbon less than R₉ and R₁₀ respectively.

3. A compound of the formula 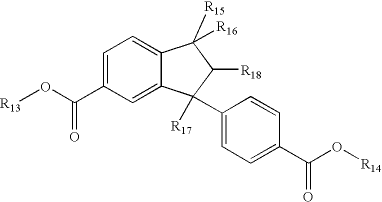
wherein
R₁₃ and R₁₄ are independently 2-acryloyloxyethyl; 2-methacryloyloxyethyl; 2-acryloyloxypropyl; 2-methacryloyloxypropyl; 3-acryloyloxy-2,2-dimethyl propyl; 3-methacryloyloxy-2,2-dimethylpropyl; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[3-(2,2-dimethylpropoxy)carbonylamino]ethyl acrylate; 2-[3-(2,2-dimethylpropoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₁₅, R₁₆, and R₁₇ are independently H, CH₃, alkyl, or aralkyl such that the carbon atom attached to the cyclopentane ring is aliphatic with at least one H;
R₁₈ is H, CH₃, alkyl, or aralkyl, containing one carbon less than R₁₅, R₁₆, or R₁₇.

4. A compound of the formula 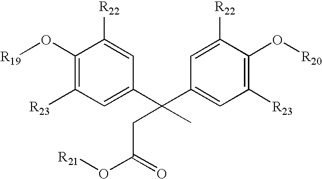
wherein
R₁₉ and R₂₀ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₂₁, R₂₂, and R₂₃ are independently H, CH₃, alkyl, or aralkyl.

5. A compound of the formula 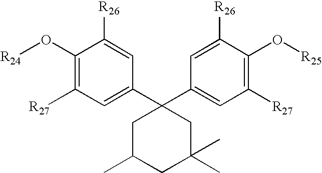
wherein
R₂₄ and R₂₅ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₂₆ and R₂₇ are independently H, CH₃, alkyl, or aralkyl;
provided when R₂₆ and R₂₇ are H that R₂₄ and R₂₅ are independently 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate.

6. A compound of the formula 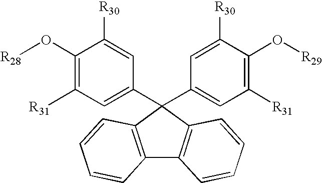
wherein
R₂₈ and R₂₉ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₃₀ and R₃₁ are independently H, CH₃, alkyl, or aralkyl;
provided when R₃₀ and R₃₁ are H or CH₃ that R₂₈ and R₂₉ are independently 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate.

7. A compound of the formula 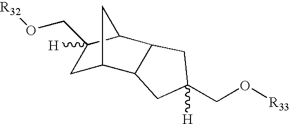
2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate.

8. A compound of the formula 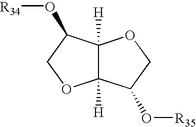
wherein R₃₄ and R₃₅ are independently 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate.

9. A compound of the formula 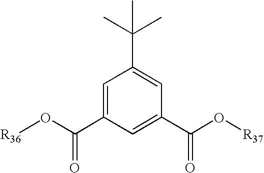
wherein R₃₆ and R₃₇ are independently 2-acryloyloxyethyl; 2-methacryloyloxyethyl; 2-acryloyloxypropyl; 2-methacryloyloxypropyl; 3-acryloyloxy-2,2-dimethylpropyl; 3-methacryloyloxy-2,2-dimethylpropyl; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[3-(2,2-dimethylpropoxy)carbonylamino]ethyl acrylate; 2-[3-(2,2-dimethylpropoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate.

10. A dental composite material comprising the compound as in any of claims 1-9.

11. A dental composite material comprising:
(a) at least one (meth)acrylic ester compound,
(b) at least one polymerization initiator compound,
(c) at least one inorganic filler, and
(d) at least one space-filling compound comprising at least one of
(e) 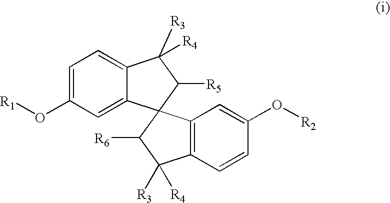
wherein
R₁ and R₂ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₃ and R₄ are independently H, CH₃, alkyl, or aralkyl such that the carbon atom attached to the cyclopentane ring is aliphatic with at least one H; and
R₅ and R₆ are independently H, CH₃, alkyl or aralkyl, containing one carbon less than R₃ and R₄ respectively;
provided when R₃ and R₄ are CH₃ and R₅ and R₆ are H that R₁ and R₂ are independently 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; 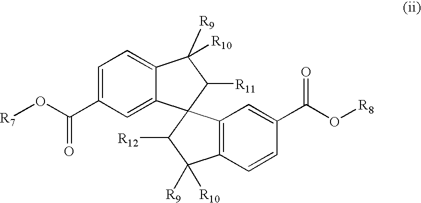
wherein
R₇ and R₈ are independently 2-acryloyloxyethyl; 2-methacryloyloxyethyl; 2-acryloyloxypropyl; 2-methacryloyloxypropyl; 3-acryloyloxy-2,2-dimethylpropyl; 3-methacryloyloxy-2,2-dimethylpropyl; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[3-(2,2-dimethylpropoxy)carbonylamino]ethyl acrylate; 2-[3-(2,2-dimethylpropoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₉ and R₁₀ are independently H, CH₃, alkyl, or aralkyl such that the carbon atom attached to the cyclopentane ring is aliphatic with at least one H;
R₁₁ and R₁₂ are independently H, CH₃, alkyl, or aralkyl, containing one carbon less than R₉ and R₁₀ respectively; 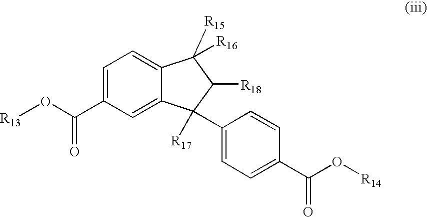
wherein
R₁₃ and R₁₄ are independently 2-acryloyloxyethyl; 2-methacryloyloxyethyl; 2-acryloyloxypropyl; 2-methacryloyloxypropyl; 3-acryloyloxy-2,2-dimethylpropyl; 3-methacryloyloxy-2,2-dimethylpropyl; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[3-(2,2-dimethylpropoxy)carbonylamino]ethyl acrylate; 2-[3-(2,2-dimethylpropoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₁₅, R₁₆, and R₁₇ are independently H, CH₃, alkyl, or aralkyl such that the carbon atom attached to the cyclopentane ring is aliphatic with at least one H;
R₁₈ is H, CH₃, alkyl, or aralkyl, containing one carbon less than R₁₅, R₁₆, or R₁₇; 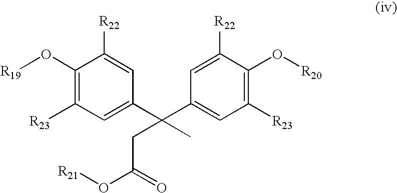
wherein
R₁₉ and R₂₀ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₂₁, R₂₂, and R₂₃ are independently H, CH₃, alkyl, or aralkyl; 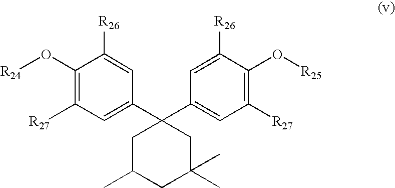
wherein
R₂₄ and R₂₅ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₂₆ and R₂₇ are independently H, CH₃, alkyl, or aralkyl;
provided when R₂₆ and R₂₇ are H that R₂₄ and R₂₅ are independently 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; 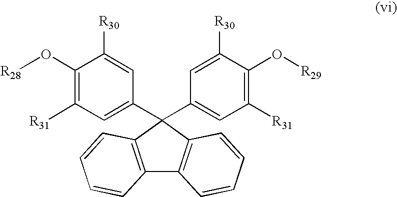
wherein
R₂₈ and R₂₉ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₃₀ and R₃₁ are independently H, CH₃, alkyl, or aralkyl;
provided when R₃₀ and R₃₁ are H or CH₃ that R₂₈ and R₂₉ are independently 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; 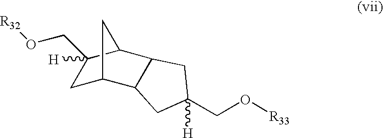
2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; 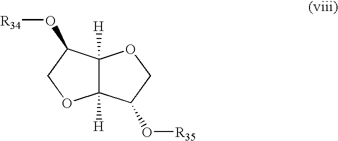
wherein R₃₄ and R₃₅ are independently 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; and 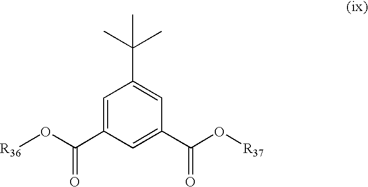
wherein R₃₆ and R₃₇ are independently 2-acryloyloxyethyl; 2-methacryloyloxyethyl; 2-acryloyloxypropyl; 2-methacryloyloxypropyl; 3-acryloyloxy-2,2-dimethylpropyl; 3-methacryloyloxy-2,2-dimethylpropyl; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[3-(2,2-dimethylpropoxy)carbonylamino]ethyl acrylate; 2-[3-(2,2-dimethylpropoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate.

12. The dental composite material of claim 11, wherein the at least one (meth)acrylic ester compound is present in an amount of from about 1 to about 99 weight percent, the at least one polymerization initiator is present in an amount of from about 0.1 to about 5 weight percent, the at least one inorganic filler is present in an amount of from about 20 to about 90 weight percent, and the at least one space-filling compound is present in an amount of from about 1 to about 100 weight percent.

13. The dental composite material of claim 11 further comprising at least one of a photoinitiating accelerator, an activator, a pigment, a radiopaquing agent, a stabilizer, and an antioxidant.

14. A dental composite material comprising at least one space-filling compound functionally terminated with at least two urethane (meth)acrylate groups.

15. The dental composite material of claim 14, wherein the at least two urethane (meth)acrylate groups independently are selected from 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; 2-(omega-polyoxypropylenecarbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[3-(2,2-dimethylpropoxy)carbonylamino]ethyl acrylate; and 2-[3-(2,2-dimethylpropoxy)carbonylamino]ethyl methacrylate.

16. A method for producing a dental restoration article with reduced shrinkage, comprising the steps of:
(a) mixing at least one space-filling compound with at least one of
(i) at least one (meth)acrylic ester compound,
(ii) at least one polymerization initiator, and
(iii) at least one inorganic filler; and
(b) forming and curing the dental restoration article;
wherein the space-filling compound comprises at least one of 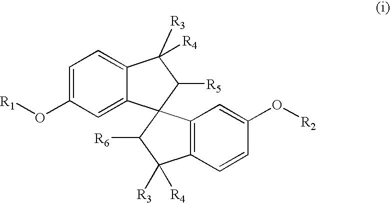
wherein
R₁ and R₂ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₃ and R₄ are independently H, CH₃, alkyl, or aralkyl such that the carbon atom attached to the cyclopentane ring is aliphatic with at least one H; and
R₅ and R₆ are independently H, CH₃, alkyl or aralkyl, containing one carbon less than R₃ and R₄ respectively;
provided when R₃ and R₄ are CH₃ and R₅ and R₆ are H that R₁ and R₂ are independently 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; 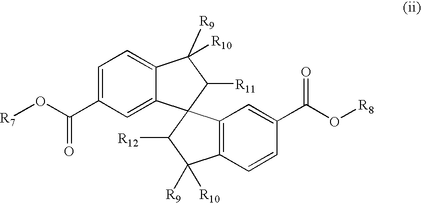
wherein
R₇ and R₈ are independently 2-acryloyloxyethyl; 2-methacryloyloxyethyl; 2-acryloyloxypropyl; 2-methacryloyloxypropyl; 3-acryloyloxy-2,2-dimethylpropyl; 3-methacryloyloxy-2,2-dimethyl propyl; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[3-(2,2-dimethylpropoxy)carbonylamino]ethyl acrylate; 2-[3-(2,2-dimethylpropoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₉ and R₁₀ are independently H, CH₃, alkyl, or aralkyl such that the carbon atom attached to the cyclopentane ring is aliphatic with at least one H;
R₁₁ and R₁₂ are independently H, CH₃, alkyl, or aralkyl, containing one carbon less than R₉ and R₁₀ respectively; 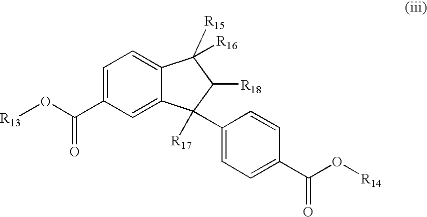
wherein
R₁₃ and R₁₄ are independently 2-acryloyloxyethyl; 2-methacryloyloxyethyl; 2-acryloyloxypropyl; 2-methacryloyloxypropyl; 3-acryloyloxy-2,2-dimethylpropyl; 3-methacryloyloxy-2,2-dimethylpropyl; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[3-(2,2-dimethylpropoxy)carbonylamino]ethyl acrylate; 2-[3-(2,2-dimethylpropoxy)carbonylamino]ethyl methacrylate; 2-[2-(2-ethoxy)ethoxycarbonylamino]ethyl acryl ate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₁₅, R₁₆, and R₁₇ are independently H, CH₃, alkyl, or aralkyl such that the carbon atom attached to the cyclopentane ring is aliphatic with at least one H;
R₁₈ is H, CH₃, alkyl, or aralkyl, containing one carbon less than R₁₅, R₁₆, or R₁₇; 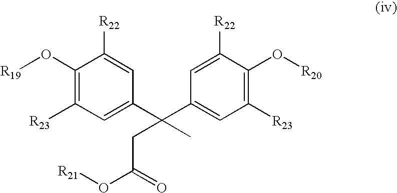
wherein
R₁₉ and R₂₀ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₂₁, R₂₂, and R₂₃ are independently H, CH₃, alkyl, or aralkyl; 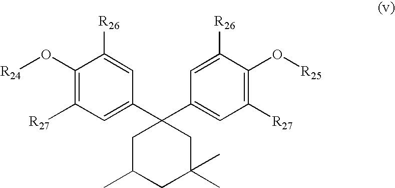
wherein
R₂₄ and R₂₅ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₂₆ and R₂₇ are independently H, CH₃, alkyl, or aralkyl;
provided when R₂₆ and R₂₇ are H that R₂₄ and R₂₅ are independently 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; 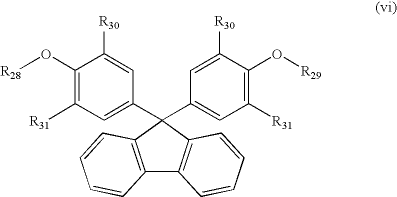
wherein
R₂₈ and R₂₉ are independently acryloyl; methacryloyl; 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate;
R₃₀ and R₃₁ are independently H, CH₃, alkyl, or aralkyl;
provided when R₃₀ and R₃₁ are H or CH₃ that R₂₈ and R₂₉ are independently 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; 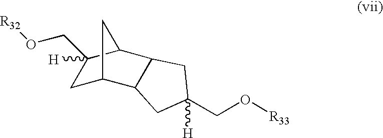
2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; 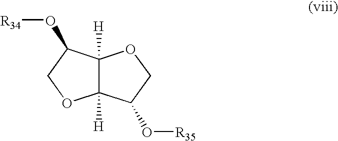
wherein R₃₄ and R₃₅ are independently 2-acryloyloxyethyl; 2-methacryloyloxyethyl; 2-acryloyloxypropyl; 2-methacryloyloxypropyl; 2-(carbonylamino)ethyl acrylate; 2-(carbonylamino)ethyl methacrylate; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate; and 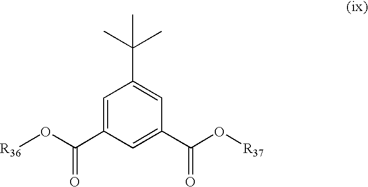
wherein R₃₆ and R₃₇ are independently 2-acryloyloxyethyl; 2-methacryloyloxyethyl; 2-acryloyloxypropyl; 2-methacryloyloxypropyl; 3-acryloyloxy-2,2-dimethylpropyl; 3-methacryloyloxy-2,2-dimethylpropyl; 2-(2-ethoxycarbonylamino)ethyl acrylate; 2-(2-ethoxycarbonylamino)ethyl methacrylate; 2-[1-(2-propoxy)carbonylamino]ethyl acrylate; 2-[1-(2-propoxy)carbonylamino]ethyl methacrylate; 2-[3-(2,2-dimethylpropoxy)carbonylamino]ethyl acrylate; 2-[3-(2,2-dimethylpropoxy)carbonylamino]ethyl methacrylate; 2-[2-(2-ethoxy)ethoxycarbonylamino]ethyl acrylate; 2-[2-(2-ethoxy)ethoxycarbonylamino]ethyl methacrylate; 2-(omega-polyoxyethylenecarbonylamino)ethyl acrylate; 2-(omega-polyoxyethylenecarbonylamino)ethyl methacrylate; 2-(omega-polyoxypropylenecarbonylamino)ethyl acrylate; or 2-(omega-polyoxypropylenecarbonylamino)ethyl methacrylate.

17. The method of claim 16, wherein the at least one (meth)acrylic ester compound is present in an amount of from about 1 to about 99 weight percent, the at least one polymerization initiator is present in an amount of from about 0.1 to about 5 weight percent, the at least one inorganic filler is present in an amount of from about 20 to about 90 weight percent, and the at least one space-filling compound is present in an amount of from about 1 to about 100 weight percent.

18. The method of claim 16, wherein the dental restoration article further comprises at least one of a photoinitiating accelerator, an activator, a pigment, a radiopaquing agent, a stabilizer, and an antioxidant.

19. The method of claim 16, wherein the dental restoration article is selected from fillings, artificial teeth, bridges, crowns, inlays, onlays, laminate veneers, facings, pit sealants, fissure sealants, cements, denture base materials, denture reline materials, orthodontic splint materials, and adhesives for orthodontic appliances.

20. A dental filling comprising the compound as in any of claims 1-9.

21. A dental inlay, onlay, facing, or laminate veneer comprising the compound as in any of claims 1-9.

22. A dental crown, bridge, or orthodontic splint material comprising the compound as in any of claims 1-9.

23. A dental adhesive, cement, sealant, or an adhesive for orthodontic appliances comprising the compound as in any of claims 1-9.

24. An artificial tooth, denture base, or denture reline material comprising the compound as in any of claims 1-9.

25. A method of treating dental tissue with a direct composite, comprising the steps of:
(a) placing a dental composite material comprising the compound as in any of claims 1-9 on a dental tissue;
(b) curing the dental composite material; and
(c) shaping the dental composite material.
